(19) United States
(12) Patent Application Publication
(10) Pub. No.: US 2026/0083322 A1
(43) Pub. Date: Mar. 26, 2026

(54) SELF-GUIDED VISION TEST USING MOBILE DEVICE

(71) Applicant: Apple Inc., Cupertino, CA (US)

(72) Inventors: Ioana NEGOITA, Sunnyvale, CA (US); Ian PERRY, San Jose, CA (US); Finnegan N. SINCLAIR, Seattle, WA (US); Trent A. GREENE, Santa Clara, CA (US); Brian W. TEMPLE, Santa Clara, CA (US)

(21) Appl. No.: 19/318,203

(22) Filed: Sep. 3, 2025

Related U.S. Application Data
(60) Provisional application No. 63/697,414, filed on Sep. 20, 2024.

(51) Int. Cl. A61B 3/113; A61B 3/14; G06F 3/01; G06F 3/0346; G06T 7/00; G06T 7/20; G06T 7/70

(52) U.S. Cl. CPC A61B 3/113; A61B 3/14; G06F 3/013; G06F 3/0346; G06T 7/0012; G06T 7/20; G06T 7/70; G06T 2207/30041

(57) ABSTRACT

A method for a self-guided vision test can include, at a first electronic device in communication with a second electronic device, obtaining data indicative of a position of a target displayed by the second electronic device relative to the first electronic device or a user of the first electronic device while the target moves relative to the first electronic device, tracking, using one or more first image sensors, a direction of gaze of the user, and based on a relationship between the position of the target and the direction of gaze while the target moves relative to the first electronic device or a user of the first electronic device, presenting an indication of abnormal eye movement.

### CROSS-REFERENCE TO RELATED APPLICATIONS

This application claims the benefit of U.S. Provisional Application No. 63/697,414, filed Sep. 20, 2024, the content of which is hereby incorporated herein by reference in its entirety for all purpose.

### FIELD OF THE DISCLOSURE

This relates generally to systems and methods for self-guided tests, and more particularly, to conducting self-guided visions tests using a head-mounted device and a mobile device.

### BACKGROUND OF THE DISCLOSURE

Some computer platforms that provide eye tracking for navigating two-dimensional and/or three-dimensional environments can be employed for self-guided tests. In some examples, a head-mounted device is adapted for a self-guided vision test using a mobile device.

### SUMMARY OF THE DISCLOSURE

Some examples of the disclosure are directed to systems and methods for a self-guided vision test. In some examples, the system of the present disclosure can include a first electronic device (e.g., a head-mounted display) having one or more first image sensors to track movement of the eye(s) of a user of the first electronic device. The system can further include a second electronic device (e.g., a mobile device) having one or more second image sensors and a display. The second electronic device can be moved across the field of view of the user while displaying a visual target for the user. As the user follows the motion of the visual target displayed by the second electronic device, the system can track a direction of gaze of the user using the one or more first image sensors on the first electronic device (e.g., the head-mounted display). The system can further track the position of the second electronic device and/or visual target relative to the first electronic device and/or the user using the one or more second image sensors of the second electronic device. The recorded eye movement can be concurrently or subsequently compared to the recorded trajectory of the visual target to evaluate the user's ability to match eye movement to the movement of the target.

The full descriptions of these examples are provided in the Drawings and the Detailed Description, and it is understood that this Summary does not limit the scope of the disclosure in any way.

### BRIEF DESCRIPTION OF THE DRAWINGS

For improved understanding of the various examples described herein, reference should be made to the Detailed Description below along with the following drawings. Like reference numerals often refer to corresponding parts throughout the drawings.

FIG. 1 illustrates an electronic device presenting a three-dimensional environment according to some examples of the disclosure.

FIGS. 2A-2B illustrate block diagrams of example architectures for electronic devices according to some examples of the disclosure.

FIGS. 3A-3B illustrate an example system for a vision test according to an example of the disclosure.

FIGS. 4A-4C illustrate an example electronic device according to an example of the disclosure.

FIGS. 5A-5B illustrate an example electronic device displaying a visual target at different distances from the head of the user.

FIG. 6 illustrates a representation of the components of the target position data as captured by the electronic device according to an example of the disclosure.

FIGS. 7A-7C illustrate example representations of a position of an example object in the field of view of the user according to an example of the disclosure.

FIG. 8 illustrates an example of a head-mounted display (HMD) according to an example of the disclosure.

FIG. 9 illustrates example representations of the data recorded by the HMD during the test or the HMD data according to an example of the disclosure.

FIG. 10 illustrates an example of a user performing a vision test according to an example of the disclosure.

FIG. 11 illustrates an example flowchart of a method of performing a vision test according to an example of the disclosure.

### DETAILED DESCRIPTION

Some examples of the disclosure are directed to systems and methods for a self-guided vision test. In some examples, the system of the present disclosure can include a first electronic device (e.g., a head-mounted display) having one or more first image sensors to track movement of the eye(s) of a user of the first electronic device. The system can further include a second electronic device (e.g., a mobile device) having one or more second image sensors and a display. The second electronic device can be moved across the field of view of the user while displaying a visual target for the user. As the user follows the motion of the visual target displayed by the second electronic device, the system can track a direction of gaze of the user using the one or more first image sensors on the first electronic device (e.g., the head-mounted display). The system can further track the position of the second electronic device and/or visual target relative to first electronic device and/or the user using the one or more second image sensors of the second electronic device. The recorded eye movement can be concurrently or subsequently compared to the recorded trajectory of the visual target to evaluate the user's ability to match eye movement to the movement of the target.

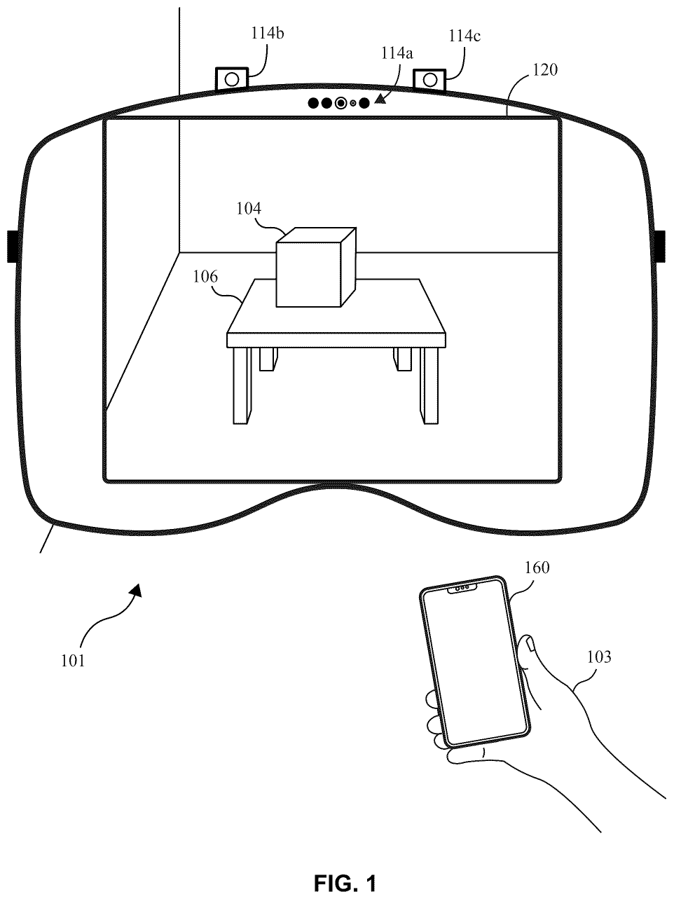

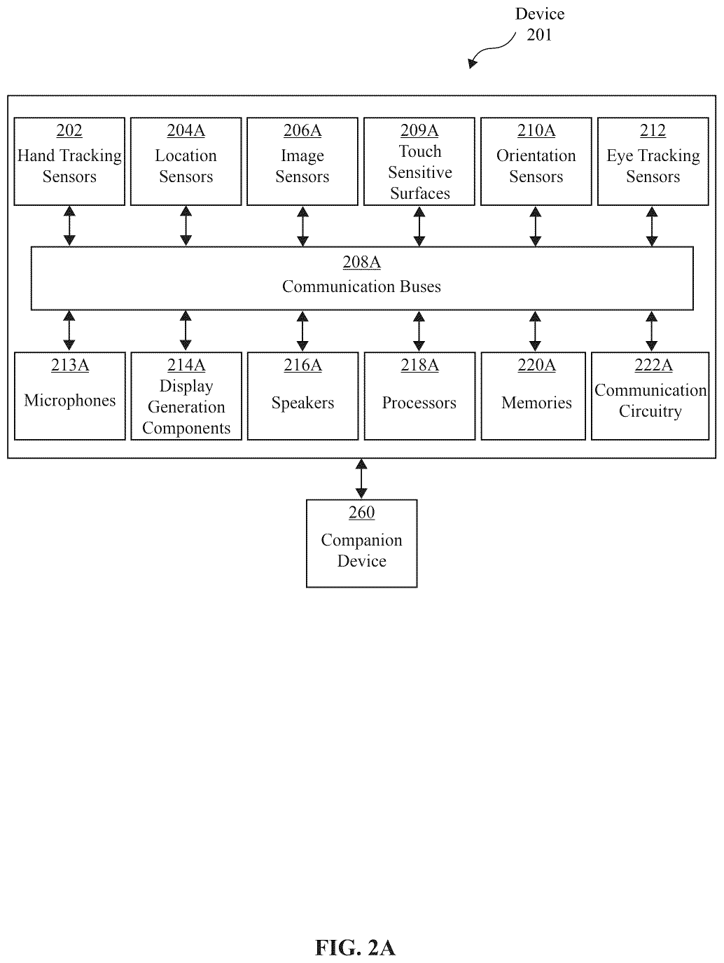

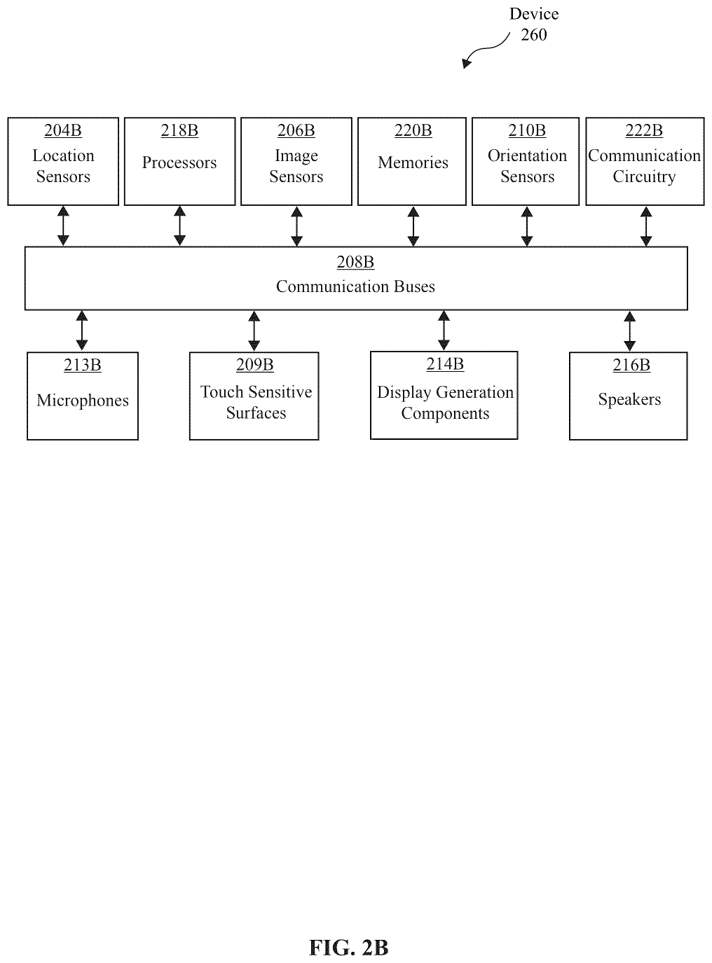

FIG. 1 illustrates an electronic device 101 presenting three-dimensional environment (e.g., an extended reality (XR) environment or a computer-generated reality (CGR) environment, optionally including representations of physical and/or virtual objects), according to some examples of the disclosure. In some examples, as shown in FIG. 1, electronic device 101 is a head-mounted display or other head-mountable device configured to be worn on a head of a user of the electronic device 101. Examples of electronic device 101 are described below with reference to the architecture block diagram of FIG. 2A. As shown in FIG. 1, electronic device 101 and table 106 are located in a physical environment. The physical environment may include physical features such as a physical surface (e.g., floor, walls) or a physical object (e.g., table, lamp, etc.). In some examples, electronic device 101 may be configured to detect and/or capture images of the physical environment including table 106 (illustrated in the field of view of electronic device 101).

In some examples, as shown in FIG. 1, electronic device 101 includes one or more internal image sensors 114a oriented towards a face of the user (e.g., eye tracking cameras as described below with reference to FIGS. 2A-2B). In some examples, internal image sensors 114a are used for eye tracking (e.g., detecting a gaze of the user). Internal image sensors 114a are optionally arranged on the left and right portions of display 120 to enable eye tracking of the user's left and right eyes. In some examples, electronic device 101 also includes external image sensors 114b and 114c facing outwards from the user to detect and/or capture the physical environment of the electronic device 101 and/or movements of the user's hands or other body parts.

In some examples, display 120 has a field of view visible to the user. In some examples, the field of view visible to the user is the same as a field of view of external image sensors 114b and 114c. For example, when display 120 is optionally part of a head-mounted device, the field of view of display 120 is optionally the same as or similar to the field of view of the user's eyes. In some examples, the field of view visible to the user is different from a field of view of external image sensors 114b and 114c (e.g., narrower than the field of view of external image sensors 114b and 114c). In other examples, the field of view of display 120 may be smaller than the field of view of the user's eyes. A viewpoint of a user determines what content is visible in the field of view, a viewpoint generally specifies a location and a direction relative to the three-dimensional environment. As the viewpoint of a user shifts, the field of view of the three-dimensional environment will also shift accordingly. In some examples, electronic device 101 may be an optical see-through device in which display 120 is a transparent or translucent display through which portions of the physical environment may be directly viewed. In some examples, display 120 may be included within a transparent lens and may overlap all or a portion of the transparent lens. In other examples, electronic device may be a video-passthrough device in which display 120 is an opaque display configured to display images of the physical environment using images captured by external image sensors 114b and 114c. While a single display is shown in FIG. 1, it is understood that display 120 optionally includes more than one display. For example, display 120 optionally includes a stereo pair of displays (e.g., left and right display panels for the left and right eyes of the user, respectively) having displayed outputs that are merged (e.g., by the user's brain) to create the view of the content shown in FIG. 1. In some examples, as discussed in more detail below with reference to FIGS. 2A-2B, the display 120 includes or corresponds to a transparent or translucent surface (e.g., a lens) that is not equipped with display capability (e.g., and is therefore unable to generate and display the virtual object 104) and alternatively presents a direct view of the physical environment in the user's field of view (e.g., the field of view of the user's eyes).

In some examples, the electronic device 101 is configured to display (e.g., in response to a trigger) a virtual object 104 in the three-dimensional environment. Virtual object 104 is represented by a cube illustrated in FIG. 1, which is not present in the physical environment, but is displayed in the three-dimensional environment positioned on the top of table 106 (e.g., real-world table or a representation thereof). Optionally, virtual object 104 is displayed on the surface of the table 106 in the three-dimensional environment displayed via the display 120 of the electronic device 101 in response to detecting the planar surface of table 106 in the physical environment 100.

It is understood that virtual object 104 is a representative virtual object and one or more different virtual objects (e.g., of various dimensionality such as two-dimensional or other three-dimensional virtual objects) can be included and rendered in a three-dimensional environment. For example, the virtual object can represent an application or a user interface displayed in the three-dimensional environment. In some examples, the virtual object can represent content corresponding to the application and/or displayed via the user interface in the three-dimensional environment. In some examples, the virtual object 104 is optionally configured to be interactive and responsive to user input (e.g., air gestures, such as air pinch gestures, air tap gestures, and/or air touch gestures), such that a user may virtually touch, tap, move, rotate, or otherwise interact with, the virtual object 104.

As discussed herein, one or more air pinch gestures performed by a user (e.g., with hand 103 in FIG. 1) are detected by one or more input devices of electronic device 101 and interpreted as one or more user inputs directed to content displayed by electronic device 101. Additionally or alternatively, in some examples, the one or more user inputs interpreted by the electronic device 101 as being directed to content displayed by electronic device 101 (e.g., the virtual object 104) are detected via one or more hardware input devices (e.g., controllers, touch pads, proximity sensors, buttons, sliders, knobs, etc.) rather than via the one or more input devices that are configured to detect air gestures, such as the one or more air pinch gestures, performed by the user. Such depiction is intended to be exemplary rather than limiting; the user optionally provides user inputs using different air gestures and/or using other forms of input.

In some examples, the electronic device 101 may be configured to communicate with a second electronic device, such as a companion device. For example, as illustrated in FIG. 1, the electronic device 101 is optionally in communication with electronic device 160. In some examples, electronic device 160 corresponds to a mobile electronic device, such as a smartphone, a tablet computer, a smart watch, a laptop computer, or other electronic device. In some examples, electronic device 160 corresponds to a non-mobile electronic device, which is generally stationary and not easily moved within the physical environment (e.g., desktop computer, server, etc.). Additional examples of electronic device 160 are described below with reference to the architecture block diagram of FIG. 2B. In some examples, the electronic device 101 and the electronic device 160 are associated with a same user. For example, in FIG. 1, the electronic device 101 may be positioned on (e.g., mounted to) a head of a user and the electronic device 160 may be positioned near electronic device 101, such as in a hand 103 of the user (e.g., the hand 103 is holding the electronic device 160), a pocket or bag of the user, or a surface near the user. The electronic device 101 and the electronic device 160 are optionally associated with a same user account of the user (e.g., the user is logged into the user account on the electronic device 101 and the electronic device 160). Additional details regarding the communication between the electronic device 101 and the electronic device 160 are provided below with reference to FIGS. 2A-2B.

In some examples, displaying an object in a three-dimensional environment is caused by or enables interaction with one or more user interface objects in the three-dimensional environment. For example, initiation of display of the object in the three-dimensional environment can include interaction with one or more virtual options/affordances displayed in the three-dimensional environment. In some examples, a user's gaze may be tracked by the electronic device as an input for identifying one or more virtual options/affordances targeted for selection when initiating display of an object in the three-dimensional environment. For example, gaze can be used to identify one or more virtual options/affordances targeted for selection using another selection input. In some examples, a virtual option/affordance may be selected using hand-tracking input detected via an input device in communication with the electronic device. In some examples, objects displayed in the three-dimensional environment may be moved and/or reoriented in the three-dimensional environment in accordance with movement input detected via the input device.

In the descriptions that follows, an electronic device that is in communication with one or more displays and one or more input devices is described. It is understood that the electronic device optionally is in communication with one or more other physical user-interface devices, such as a touch-sensitive surface, a physical keyboard, a mouse, a joystick, a hand tracking device, an eye tracking device, a stylus, etc. Further, as described above, it is understood that the described electronic device, display and touch-sensitive surface are optionally distributed between two or more devices. Therefore, as used in this disclosure, information displayed on the electronic device or by the electronic device is optionally used to describe information outputted by the electronic device for display on a separate display device (touch-sensitive or not). Similarly, as used in this disclosure, input received on the electronic device (e.g., touch input received on a touch-sensitive surface of the electronic device, or touch input received on the surface of a stylus) is optionally used to describe input received on a separate input device, from which the electronic device receives input information.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, a television channel browsing application, and/or a digital video player application.

FIGS. 2A-2B illustrate block diagrams of example architectures for electronic devices according to some examples of the disclosure. In some examples, electronic device 201 and/or electronic device 260 include one or more electronic devices. For example, the electronic device 201 may be a portable device, an auxiliary device in communication with another device, a head-mounted display, a head-worn speaker, etc., respectively. In some examples, electronic device 201 corresponds to electronic device 101 described above with reference to FIG. 1. In some examples, electronic device 260 corresponds to electronic device 160 described above with reference to FIG. 1.

As illustrated in FIG. 2A, the electronic device 201 optionally includes one or more sensors, such as one or more hand tracking sensors 202, one or more location sensors 204A, one or more image sensors 206A (optionally corresponding to internal image sensors 114a and/or external image sensors 114b and 114c in FIG. 1), one or more touch-sensitive surfaces 209A, one or more motion and/or orientation sensors 210A, one or more eye tracking sensors 212, one or more microphones 213A or other audio sensors, one or more body tracking sensors (e.g., torso and/or head tracking sensors), etc. The electronic device 201 optionally includes one or more output devices, such as one or more display generation components 214A, optionally corresponding to display 120 in FIG. 1, one or more speakers 216A, one or more haptic output devices (not shown), etc. The electronic device 201 optionally includes one or more processors 218A, one or more memories 220A, and/or communication circuitry 222A. One or more communication buses 208A are optionally used for communication between the above-mentioned components of electronic device 201.

Additionally, the electronic device 260 optionally includes the same or similar components as the electronic device 201. For example, as shown in FIG. 2B, the electronic device 260 optionally includes one or more location sensors 204B, one or more image sensors 206B, one or more touch-sensitive surfaces 209B, one or more orientation sensors 210B, one or more microphones 213B, one or more display generation components 214B, one or more speakers 216B, one or more processors 218B, one or more memories 220B, and/or communication circuitry 222B. One or more communication buses 208B are optionally used for communication between the above-mentioned components of electronic device 260.

The electronic devices 201 and 260 are optionally configured to communicate via a wired or wireless connection (e.g., via communication circuitry 222A, 222B) between the two electronic devices. For example, as indicated in FIG. 2A, the electronic device 260 may function as a companion device to the electronic device 201. For example, in some examples, the electronic device 260 processes sensor inputs from electronic devices 201 and 260 and/or generates content for display using display generation components 214A of electronic device 201.

Communication circuitry 222A, 222B optionally includes circuitry for communicating with electronic devices, networks, such as the Internet, intranets, a wired network and/or a wireless network, cellular networks, and wireless local area networks (LANs). Communication circuitry 222A, 222B optionally includes circuitry for communicating using near-field communication (NFC) and/or short-range communication, such as Bluetooth®, etc. In some examples, communication circuitry 222A, 222B includes or supports Wi-Fi (e.g., an 802.11 protocol), Ethernet, ultra-wideband (“UWB”), high frequency systems (e.g., 900 MHz, 2.4 GHz, and 5.6 GHz communication systems), or any other communications protocol, or any combination thereof.

One or more processors 218A, 218B include one or more general processors, one or more graphics processors, and/or one or more digital signal processors. In some examples, one or more processors 218A, 218B include one or more microprocessors, one or more central processing units, one or more application-specific integrated circuits, one or more field-programmable gate arrays, one or more programmable logic devices, or a combination of such devices. In some examples, memories 220A and/or 220B are a non-transitory computer-readable storage medium (e.g., flash memory, random access memory, or other volatile or non-volatile memory or storage) that stores computer-readable instructions configured to be executed by the one or more processors 218A, 218B to perform the techniques, processes, and/or methods described herein. In some examples, memories 220A and/or 220B can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on compact disc (CD), digital versatile disc (DVD), or Blu-ray technologies, as well as persistent solid-state memory such as flash, solid-state drives, and the like.

In some examples, one or more display generation components 214A, 214B include a single display (e.g., a liquid-crystal display (LCD), organic light-emitting diode (OLED), or other types of display). In some examples, the one or more display generation components 214A, 214B include multiple displays. In some examples, the one or more display generation components 214A, 214B can include a display with touch capability (e.g., a touch screen), a projector, a holographic projector, a retinal projector, a transparent or translucent display, etc. In some examples, the electronic device does not include one or more display generation components 214A or 214B. For example, instead of the one or more display generation components 214A or 214B, some electronic devices include transparent or translucent lenses or other surfaces that are not configured to display or present virtual content. However, it should be understood that, in such instances, the electronic device 201 and/or the electronic device 260 are optionally equipped with one or more of the other components illustrated in FIGS. 2A and 2B and described herein, such as the one or more hand tracking sensors 202, one or more eye tracking sensors 212, one or more image sensors 206A, and/or the one or more motion and/or orientations sensors 210A. Alternatively, in some examples, the one or more display generation components 214A or 214B are provided separately from the electronic devices 201 and/or 260. For example, the one or more display generation components 214A, 214B are in communication with the electronic device 201 (and/or electronic device 260), but are not integrated with the electronic device 201 and/or electronic device 260 (e.g., within a housing of the electronic devices 201, 260). In some examples, electronic devices 201 and 260 include one or more touch-sensitive surfaces 209A and 209B, respectively, for receiving user inputs, such as tap inputs and swipe inputs or other gestures (e.g., hand-based or finger-based gestures). In some examples, the one or more display generation components 214A, 214B and the one or more touch-sensitive surfaces 209A, 209B form one or more touch-sensitive displays (e.g., a touch screen integrated with each of electronic devices 201 and 260 or external to each of electronic devices 201 and 260 that is in communication with each of electronic devices 201 and 260).

Electronic devices 201 and 260 optionally include one or more image sensors 206A and 206B, respectively. The one or more image sensors 206A, 206B optionally include one or more visible light image sensors, such as charged coupled device (CCD) sensors, and/or complementary metal-oxide-semiconductor (CMOS) sensors operable to obtain images of physical objects from the real-world environment. The one or more image sensors 206A, 206B also optionally include one or more infrared (IR) sensors, such as a passive or an active IR sensor, for detecting infrared light from the real-world environment. For example, an active IR sensor includes an IR emitter for emitting infrared light into the real-world environment. The one or more image sensors 206A, 206B also optionally include one or more cameras configured to capture movement of physical objects in the real-world environment. The one or more image sensors 206A, 206B also optionally include one or more depth sensors configured to detect the distance of physical objects from electronic device 201, 260. In some examples, information from one or more depth sensors can allow the device to identify and differentiate objects in the real-world environment from other objects in the real-world environment. In some examples, one or more depth sensors can allow the device to determine the texture and/or topography of objects in the real-world environment. In some examples, the one or more image sensors 206A or 206B are included in an electronic device different from the electronic devices 201 and/or 260. For example, the one or more image sensors 206A, 206B are in communication with the electronic device 201, 260, but are not integrated with the electronic device 201, 260 (e.g., within a housing of the electronic device 201, 260). Particularly, in some examples, the one or more cameras of the one or more image sensors 206A, 206B are integrated with and/or coupled to one or more separate devices from the electronic devices 201 and/or 260 (e.g., but are in communication with the electronic devices 201 and/or 260), such as one or more input and/or output devices (e.g., one or more speakers and/or one or more microphones, such as earphones or headphones) that include the one or more image sensors 206A, 206B. In some examples, electronic device 201 or electronic device 260 corresponds to a head-worn speaker (e.g., headphones or earbuds). In such instances, the electronic device 201 or the electronic device 260 is equipped with a subset of the other components illustrated in FIGS. 2A and 2B and described herein. In some such examples, the electronic device 201 or the electronic device 260 is equipped with one or more image sensors 206A, 206B, the one or more motion and/or orientations sensors 210A, 210B, and/or speakers 216A, 216B.

In some examples, electronic device 201, 260 uses CCD sensors, event cameras, and depth sensors in combination to detect the physical environment around electronic device 201, 260. In some examples, the one or more image sensors 206A, 206B include a first image sensor and a second image sensor. The first image sensor and the second image sensor work in tandem and are optionally configured to capture different information of physical objects in the real-world environment. In some examples, the first image sensor is a visible light image sensor, and the second image sensor is a depth sensor. In some examples, electronic device 201, 260 uses the one or more image sensors 206A, 206B to detect the position and orientation of electronic device 201, 260 and/or the one or more display generation components 214A, 214B in the real-world environment. For example, electronic device 201, 260 uses the one or more image sensors 206A, 206B to track the position and orientation of the one or more display generation components 214A, 214B relative to one or more fixed objects in the real-world environment.

In some examples, electronic devices 201 and 260 include one or more microphones 213A and 213B, respectively, or other audio sensors. Electronic device 201, 260 optionally uses the one or more microphones 213A, 213B to detect sound from the user and/or the real-world environment of the user. In some examples, the one or more microphones 213A, 213B include an array of microphones (e.g., a plurality of microphones) that optionally operate in tandem, such as to identify ambient noise or to locate the source of sound in space of the real-world environment.

Electronic devices 201 and 260 include one or more location sensors 204A and 204B, respectively, for detecting a location of electronic device 201 and/or the one or more display generation components 214A and a location of electronic device 260 and/or the one or more display generation components 214B, respectively. For example, the one or more location sensors 204A, 204B can include a global positioning system (GPS) receiver that receives data from one or more satellites and allows electronic device 201, 260 to determine the absolute position of the electronic device in the physical world.

Electronic devices 201 and 260 include one or more orientation sensors 210A and 210B, respectively, for detecting orientation and/or movement of electronic device 201 and/or the one or more display generation components 214A and orientation and/or movement of electronic device 260 and/or the one or more display generation components 214B, respectively. For example, electronic device 201, 260 uses the one or more orientation sensors 210A, 210B to track changes in the position and/or orientation of electronic device 201, 260 and/or the one or more display generation components 214A, 214B, such as with respect to physical objects in the real-world environment. The one or more orientation sensors 210A, 210B optionally include one or more gyroscopes and/or one or more accelerometers.

Electronic device 201 includes one or more hand tracking sensors 202 and/or one or more eye tracking sensors 212, in some examples. It is understood, that although referred to as hand tracking or eye tracking sensors, that electronic device 201 additionally or alternatively optionally includes one or more other body tracking sensors, such as one or more leg, one or more torso and/or one or more head tracking sensors. The one or more hand tracking sensors 202 are configured to track the position and/or location of one or more portions of the user's hands, and/or motions of one or more portions of the user's hands with respect to the three-dimensional environment, relative to the one or more display generation components 214A, and/or relative to another defined coordinate system. The one or more eye tracking sensors 212 are configured to track the position and movement of a user's gaze (e.g., a user's attention, including eyes, face, or head, more generally) with respect to the real-world or three-dimensional environment and/or relative to the one or more display generation components 214A. In some examples, the one or more hand tracking sensors 202 and/or the one or more eye tracking sensors 212 are implemented together with the one or more display generation components 214A. In some examples, the one or more hand tracking sensors 202 and/or the one or more eye tracking sensors 212 are implemented separate from the one or more display generation components 214A. In some examples, electronic device 201 alternatively does not include the one or more hand tracking sensors 202 and/or the one or more eye tracking sensors 212. In some such examples, the one or more display generation components 214A may be utilized by the electronic device 260 to provide a three-dimensional environment and the electronic device 260 may utilize input and other data gathered via the other one or more sensors (e.g., the one or more location sensors 204A, the one or more image sensors 206A, the one or more touch-sensitive surfaces 209A, the one or more motion and/or orientation sensors 210A, and/or the one or more microphones 213A or other audio sensors) of the electronic device 201 as input and data that is processed by the one or more processors 218B of the electronic device 260. Additionally or alternatively, electronic device 260 optionally does not include other components shown in FIG. 2B, such as the one or more location sensors 204B, the one or more image sensors 206B, the one or more touch-sensitive surfaces 209B, etc. In some such examples, the one or more display generation components 214A may be utilized by the electronic device 260 to provide a three-dimensional environment and the electronic device 260 may utilize input and other data gathered via the one or more motion and/or orientation sensors 210A (and/or the one or more microphones 213A) of the electronic device 201 as input.

In some examples, the one or more hand tracking sensors 202 (and/or other body tracking sensors, such as leg, torso and/or head tracking sensors) can use the one or more image sensors 206 (e.g., one or more IR cameras, 3D cameras, depth cameras, etc.) that capture three-dimensional information from the real-world including one or more body parts (e.g., hands, legs, or torso of a human user). In some examples, the hands can be resolved with sufficient resolution to distinguish fingers and their respective positions. In some examples, the one or more image sensors 206A are positioned relative to the user to define a field of view of the one or more image sensors 206A and an interaction space in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, the one or more eye tracking sensors 212 include at least one eye tracking camera (e.g., IR cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Electronic devices 201 and 260 are not limited to the components and configuration of FIGS. 2A-2B, but can include fewer, other, or additional components in multiple configurations. In some examples, electronic device 201 and/or electronic device 260 can each be implemented between multiple electronic devices (e.g., as a system). In some such examples, each of (or more of) the electronic devices may include one or more of the same components discussed above, such as various sensors, one or more display generation components, one or more speakers, one or more processors, one or more memories, and/or communication circuitry. A person or persons using electronic device 201 and/or electronic device 260, is optionally referred to herein as a user or users of the device.

Attention is now directed towards systems and methods for conducting self-guided visions tests using a head-mounted device and a mobile device.

Pursuit tracking tests are commonly used for neurological testing. The tests aim to measure the patient's ability to track a moving target with their eyes. Such tests are primarily employed to detect cerebral conditions such as traumatic brain injury or concussions. The measurements can also help detect eye-related disorders or injuries like such as eyeball injuries or disorders with the orbital muscle, as well as neurocognitive disorders such as Parkinson's disease and Alzheimer's disease, and others. To that end, the user is directed to follow a moving target with their eyes while their eye movement is recorded to analyze the patterns of their gaze. The ability to match eye movement to the visual target's movement provides information about the patient's neurological health. Systems exist that provide a moving image or stimulus across a display as a target for a pursuit tracking test. However, movement of the target on such systems is limited by the field of view of the display, which is often narrower than the field of view of the user. In particular, certain visual tests require the target to be far or at a greater angle from the centerline of the head of the user and thus require a wider field of view than a display of an electronic device is able to provide.

Examples of the present disclosure address these disadvantages by providing solutions for performing a self-guided vision test whose visual scope extends beyond the limitations of the field of view of a display. In some examples, the system of the present disclosure includes a head-mounted display having a field of view and configured to record the movement of the eyes of the user. The system further includes an electronic device (e.g., a mobile device) having a camera to track position and a display to serve as a visual target to be tracked by the user's eyes during the test. In some examples, the electronic device displays a visual indicator to serve as a visual target for the gaze of the user during the test (e.g., for the user to focus their eye(s) upon). The electronic device is positioned in the field of view of the user with its display and camera facing the user, and the electronic device displays the visual target on the display. As the user focuses on the visual target, the device can be moved across the field of view of the user and optionally outside of the field of view of the head-mounted display. In some examples, the user holds the electronic device at arm's length and moves it relative to the head of the user while maintaining their gaze on the visual target. The system records the gaze of the eye(s) of the user using inward facing cameras on the head-mounted display while the user visually tracks the motion of the electronic device with their eyes. The system is further configured to determine relative positioning of the head of the user and the electronic device by tracking the position of the electronic device relative to the head of the user using the camera that is part of the electronic device. The recorded eye movement is concurrently or subsequently compared to the recorded target's trajectory to evaluate the user's ability to match eye movement to the target's movement.

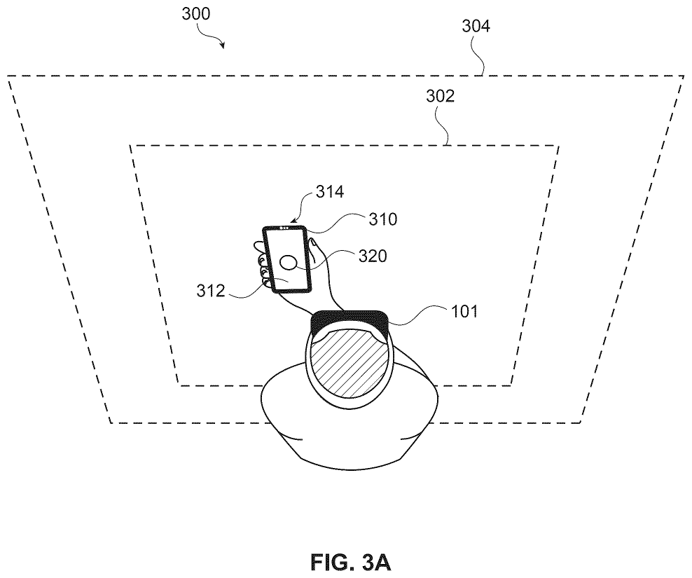

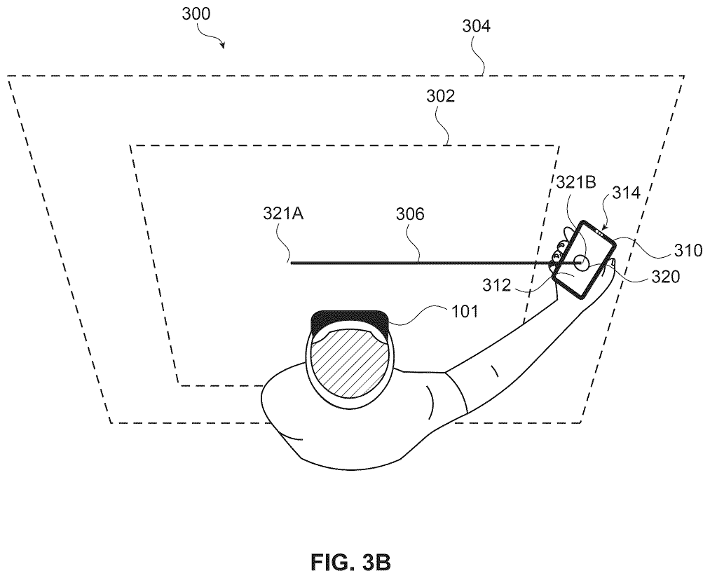

FIGS. 3A-3B illustrate an example system 300 for a vision test system according to an example of the disclosure. The system can include electronic device 101 or equivalently, a head-mounted device (HMD) 101, installed on the head of the user. In some examples, HMD 101 corresponds to electronic device 201 described above with reference to FIG. 2A. HMD 101 is configured track a direction of gaze of the user using one or more image sensors such as image sensors 206A or eye tracking sensor(s) 212 described with respect to FIG. 2A. In some examples, HMD 101 does not include a display and/or is not in communication with a display (e.g., HMD 101 is optionally a head-mounted device without a display). In some examples, HMD 101 can include a display 120 (illustrated in FIG. 1) that has a field of view 302 visible to the user. In some examples, the field of view 302 corresponds to the field of view of external image sensors 114b and 114c, previously described in reference to FIG. 1. In some examples, the field of view 302 of HMD 101 may be smaller than the field of view 304 of the user. In the examples illustrated in FIGS. 3A-3B, the field of view 304 of the user extends past all sides of the display field of view 302 and comprises both field of view 302 and the area outside of field of view 302. Field of view 302 is shown as a rectangle for illustrative purposes only and may have any number of shapes. For example, the shape of field of view 302 may be a square, an oval, or a circle.

System 300 can further include an electronic device 310 positioned within the field of view 304 of the user. In some examples, electronic device 310 corresponds to electronic device 160 described above with reference to FIG. 1 and/or electronic device 260 described above with reference to FIG. 2B. In FIGS. 3A-3B, the user is shown as holding the electronic device 310 within the field of view of the user. The electronic device 310 may have a display 312 and a camera 314. The electronic device 310 may be positioned to face the user such that the display 312 is in the field of view of the user (e.g., the user can see the display 312), and the user is in the field of view of the camera (e.g., the camera 314 can record the user). The electronic device 310 may display a target 320 on display 312. The target 320 serves as a point on the electronic device 310 to be followed as a visual target by the eyes of the user during a vision test according to examples of the disclosure. In some examples, target 320 can move relative to the user in the field of view 304 of the user, either because the target moves on the display 312 of the electronic device 310 relative to the user, the electronic device 310 itself moves relative to the user while displaying the target 320, or the target 320 moves on the display 312 of the electronic device 310 relative to the user while the electronic device 310 moves relative to the user. In some examples, the target 320 can move relative to the user according to a trajectory or a pattern that, in some examples, can be predetermined. In some examples, such as illustrated in FIGS. 3A and 3B, the user can hold the electronic device 310 (e.g., at arm's length) and move the electronic device 310 (and therefore target 320) relative to the head of the user and around the field of view of the user (e.g., in a self-guided test) from a first electronic device position such as shown in FIG. 3A, to a second electronic device position such as shown in FIG. 3B. As the user moves the electronic device 310 from the first device position to the second device position, the target displayed on the electronic device 310 has moved from a first target position 321A to a second target position 321B according to a trajectory 306. In some examples, the trajectory 306 of the target 320 displayed on the electronic device 310 can extend outside of the field of view 302 of the HMD 101 as the user moves the electronic device outside of the field of view 302 of the HMD (e.g., such that target 320 is located at position 321B). In some examples, the system 300 can instruct the user via one or more output devices (e.g., speakers of the HMD 101) on where to position and how to move the electronic device.

During the motion of the target 320 (e.g., as the user moves the electronic device 310 within the field of view of the user), the user is visually tracking the target 320 displayed on the electronic device, including when the target is located outside of the field of view 302 of the HMD 101 such as shown in FIG. 3B. While the user is tracking the target 320 with their eye(s), the HMD 101 can track the direction of gaze of the user using one or more image sensors to provide eye-tracking data indicative of the user's eye movement. In some examples, the one or more image sensors of the HMD 101 can track the direction of gaze of the user when the target is positioned past the field of view 302 of HMD 101 such as shown in FIG. 3B where the target is located at position 321B. The HMD 101 can further record head movements of the user to refine the eye-tracking data. Concurrently, while the target is moving relative to the head of the user, the electronic device 310 can record the target position data relative to the head of the user using one or more of: camera 314 to record the face of the user, an IMU of the electronic device to record inertial data of the electronic device, and information pertaining to the location of the target on the display of the electronic device. A processor can process both the target position data recorded by the electronic device and the eye-tracking data (including the direction of gaze) recorded by the HMD 101 during the motion of the target 320 in the field of view 304 of the user and determine based on whether one or more criteria are satisfied including a criterion based on a relationship between the position of the target 320 and the direction of gaze while the target 320 moves relative to the electronic device 310, whether an abnormality exists in recorded eye movement.

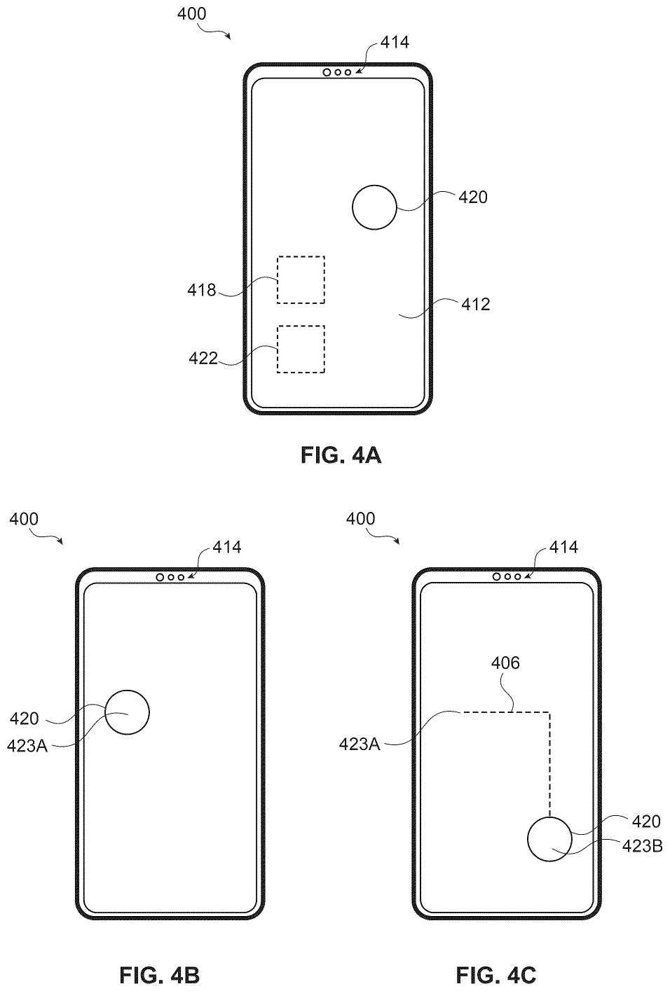

FIG. 4A-4C illustrate an example electronic device 400 according to an example of the disclosure. In some examples, electronic device 400 corresponds to electronic device 310 described above with reference to FIGS. 3A-3B. Electronic device 400 can be a mobile device in some examples. Electronic device 400 can include a display 412. In some examples, the display 412 can be a liquid-crystal display (LCD), an organic light-emitting diode (OLED), or other type of display. Electronic device 400 can display via display 412 a visual target or target 420 for the vision test enabled by system 300, in some examples. A target for the vision test can be physical (e.g., a finger, a sign on a piece of paper, the electronic device 400 itself), or a virtual object (e.g., an indicator displayed by the electronic device 400) that the user is instructed to follow with their eye(s) during the test. In the example of FIG. 4, target 420 is a circular virtual indicator displayed by the electronic device 400. As target 420 is intended to be seen by the eyes of the user and/or the HMD 101, the electronic device 400 can be oriented such that the display 412 faces the HMD 101 and/or face of the user and the target 420 is visible to the user (e.g., in the field of view 304 of the user).

The target 420 is configured to move relative to the HMD 101 and/or the head of the user (e.g., the face and/or the eyes of the user). Accordingly, in some examples, such as illustrated in FIGS. 4B and 4C, the electronic device 400 can display the target 420 as a moving indicator on the display 412 (e.g., using an animation) such that the target 420 moves around the display 412 and relative to the HMD 101 and/or the user. In some examples, the electronic device 400 can display target 420 as moving around display 412 in a predetermined manner. In some examples, target 420 can move around display 412 according to a pattern. In some examples, target 420 can move around display 412 according to an arbitrary manner (e.g., without following a pattern). In FIGS. 4B and 4C, target 420 is shown moving from a first target location 423A on the display 412 of the electronic device 400 and along trajectory 406 to a second target location 423B on the display 412 of the electronic device. In some examples, the target 420 can move relative to HMD 101 and/or head of the user because the electronic device 400 displaying the target 420 moves relative to the HMD 101 and/or the head of the user. In this case, the target 420 can be stationary relative to the display 412 while the electronic device 400 moves in the field of view 304 of the user. Alternatively, the target 420 can be displayed as moving around the display 412 while the electronic device 400 moves relative to the head of the user. In some examples, the electronic device 400 can be moved by the user, such as in illustrated in FIG. 3 where the user holds the electronic device 310 (equivalent to electronic device 400) at arm's length and moves the electronic device 310 relative to their head or to the HMD 101 while keeping the display 312 oriented toward the user and/or the HMD 101 such that target is visible to the user. While FIG. 3 shows the user moving the electronic device 310 that displays the target 320 relative to the user and/or HMD 101, it is understood that in some examples, the target 320 can be stationary and the user and/or HMD 101 can move their head while maintaining their eye(s) on the target 320. In such an example, it can be said that the target 320 moves relative to the head of the user even though the target 320 is stationary and the user is in motion, as within the field of view 304 of the user the target 320 appears to be in motion. In some examples, the target moves through the field of view 304 of the user according to a pattern.

Referring back to FIG. 4, the electronic device 400 can further include one or more image sensors 414. The image sensor 414 of the electronic device 400 may also include one or more image cameras, depth sensors, thermal (e.g., infrared) sensors, and the like. The image sensor 414 is configured to capture images. Images captured by image sensor 414 may include, for example, still images, video images, and/or frame-by-frame images. In some examples, the image sensor 414 is located on the front of the device, e.g., on the same side of the device as the display 412. In some examples, the image sensor 414 is configured to capture images of the user's face while the user is gazing at the target 420. Optionally or alternatively, in some examples, the image sensor 414 is configured to capture images of the HMD 101. Accordingly, both display 412 and the image sensor 414 are oriented toward the user such that the target 420 displayed on display 412 is visible to the user (e.g., is in the field of view 304 of the user) and the user's face is visible to the image sensor, such as camera 414 (e.g., within the image sensor's field of view). As with the orientation of the display 412 showing the target 420 to the user, the visibility of the user to the image sensor 414 may be maintained throughout the motion of the target and/or the electronic device 400 relative to the user's face and/or the HMD 101.

In some examples, the electronic device 400 can further include an inertial measurement unit (IMU) 418. The IMU 418 can include one or more orientation sensors configured to track changes in the position and/or orientation of electronic device 400 and/or display 412, such as with respect to physical objects including the HMD 101 and or head of the user. The one or more orientation sensors can include an accelerometer, a gyroscope, a compass, for example.

In some examples, the electronic device 400 can include communication circuitry 422. Communication circuitry 422 optionally includes circuitry for communicating with electronic devices, networks, such as the Internet, intranets, a wired network and/or a wireless network, cellular networks, and wireless local area networks (LANs). Communication circuitry 422 optionally includes circuitry for communicating using near-field communication (NFC) and/or short-range communication, such as Bluetooth®. The electronic device 400 can be in communication with the HMD 101 using communication circuitry 422 via one or more wired or wireless communication channels. The electronic device 400 can transmit to the HMD 101 the target position data (described in further detail below) as the target 420 moves in relation the HMD 101 via the one or more communication channels. In some examples, the electronic device can receive data recorded by the HMD 101 during the motion of the electronic device relative to the head of the user, such as direction of gaze data, HMD inertial data, and HMD target position data, as will be further detailed below.

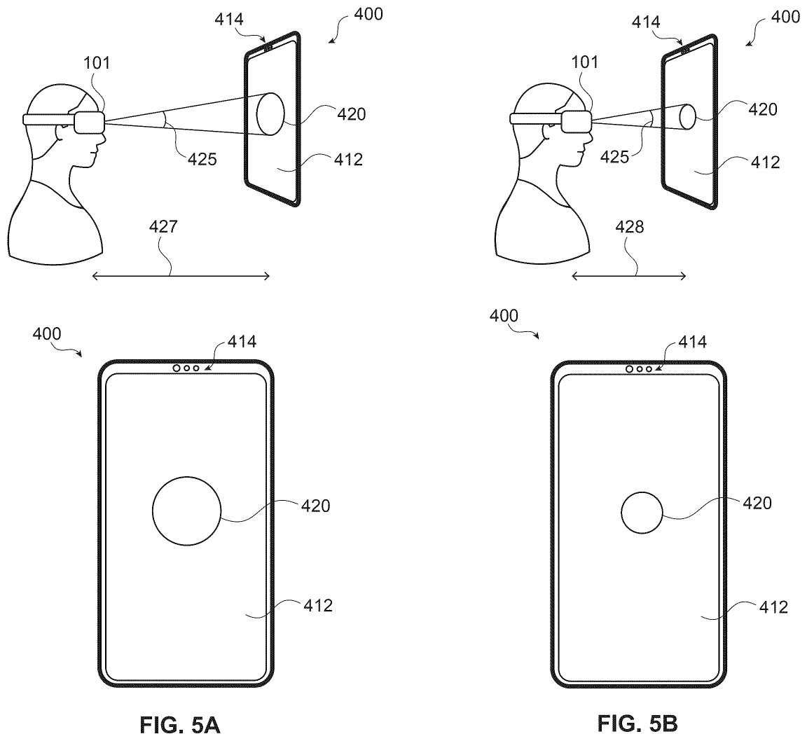

FIGS. 5A-5B illustrate an example electronic device 400 displaying target 420 at different distances from the head of the user. In some examples, the electronic device 400 can maintain an angular size of the target 420 from the perspective of the HMD 101 and/or the head of the user when a distance between the target 420 and the HMD 101 and/or the user changes. Herein, an angular size or visual size refers to the apparent size of the target 420 from the perspective of the user. In some examples, the electronic device 400 can increase or decrease the size of target 420 on display 412 based on the distance of the electronic device 400 from the head of the user to maintain an angular size of the target 420. In FIG. 5A, the target is located at a first distance 427 from the HMD 101 and/or the user and the electronic device 400 displays the target 420 at a first size and an angular size 425. In FIG. 5B, the target is located as a second distance 428 from the HMD 101 and/or the user. The second distance 428 is smaller than the first distance 427 (e.g., because the user has moved the electronic device 400 closer to their face). In response, the electronic device 400 displays the target 420 at a second size (e.g., a smaller size) on the display 412 to maintain a constant angular size 425 of the target 420 from the perspective of the HMD 101 and/user. It is understood that in FIG. 5B, if the second distance 428 were greater than the first distance 427 (e.g., because the user has moved the electronic device 400 further from their face), in response, the electronic device 400 would display target 420 a larger size on the display 412 to maintain the same (e.g., constant) angular size 425 of target 420 from the perspective of the HMD 101 and/user. In some examples, varying the size of the target 420 based on distance from the HMD 101 and/or user to maintain an angular size for the target 420 from the perspective of the HMD 101 and/or helps maintain the target 420 at a constant relative size to reduce errors in the eye-tracking test.

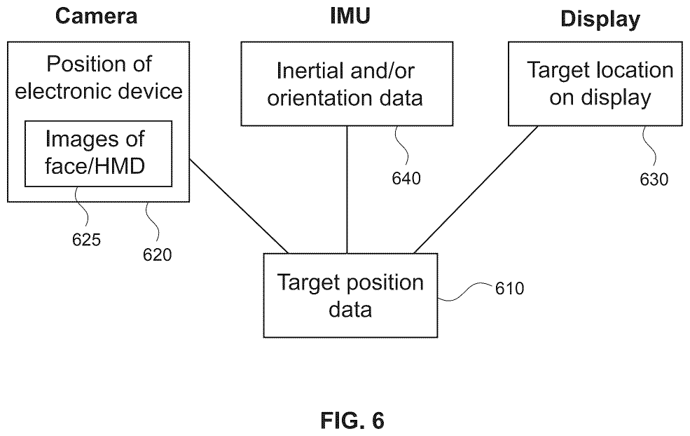

FIG. 6 illustrates a representation of the components of the target position data 610 (e.g., the data indicative of the position of the target relative to the user) as captured by the electronic device 400 according to an example of the disclosure. The target position data 610 can include electronic device position data 620, which includes the position of the electronic device 400 relative to the user and/or HMD 101. In some examples, the electronic device 400 can determine its position relative to the head of the user and/or the HMD 101 based one or more images 625 of the face of the user and/or HMD captured by the image sensor 414 of the electronic device 400. In particular, the electronic device 400 can detect and identify features of the face of the user in the one or more images to determine its position relative to the user. In some examples, image sensor 414 can include a depth sensor that enables the image sensor 414 to capture depth information in the one or more images of the face of the user to facilitate identification of the features by the electronic device 400. The identified features can include for example the eyes, nose, mouth, chin, outline of the user's face, eyebrows, and any other feature of the user's face that the electronic device 400 can be configured to detect in the one or more images. The electronic device 400 can apply facial recognition algorithms to detect the features of the user's face in the one or more images. Using the detected features, the electronic device 400 can further determine the electronic device position data 620 (e.g., the position of the electronic device 400 in relation to the head of the user). For example, the electronic device 400 can determine the position of the electronic device 400 relative to the head of the user based on a size and/or an orientation of one or more features of the detected features, and/or a distance between the one or more features of the detected features in the one or more images.

In some examples, the electronic device 400 can detect and identify physical features of the HMD 101 in the one or more images to determine its position relative to the HMD 101 and/or the face of the user. The physical features can include for example lines, shapes, angles, colors, reflections, and other identifiable features of the HMD 101 that the electronic device 400 can be configured to detect in the one or more images. The electronic device 400 can apply shape or pattern recognition algorithms to detect the physical features of the HMD 101 in the one or more images. Using the detected physical features, the electronic device 400 can further determine the position of the electronic device 400 in relation to the HMD 101 and/or the face of the user (e.g., as included in electronic device position data 620). For example, the electronic device 400 can determine the position of the electronic device 400 relative to the face of the user (e.g., electronic device position data 620) based on a size and/or an orientation of one or more physical features of the detected physical features, and/or a distance between the one or more features of the detected physical features in the one or more images.

It is understood that in some examples, the electronic device 400 can determine the position of the electronic device 400 in relation to the HMD 101 and/or the head of the user based on both the detected features of the user's face and the detected physical features of the HMD 101.

In some examples, the target position data 610 can further include the target location data 630 (e.g., the target location on the display 412). The target location on the display 412 in known to the electronic device 400 as the electronic device 400 is displaying the target. Using both electronic device position data 620 (e.g., the position of the electronic device 400 relative to the user's face) as determined based on the one or more images of the user's face as described above, and the target location data 630 (e.g., the target location on the display 412) as displayed by the electronic device 400, the electronic device 400 can derive the position of the target relative to the face of the user, and therefore the target position data 610 (e.g., relative to the face of the user). For example, the electronic device 400 can offset the position of the electronic device (e.g., electronic device position data 620) relative to the user's face (as calculated from the one or more images) by the target location on the display (known by the electronic device as target location data 630) to determine the position of the target relative to the user's face (e.g., the target position data 610).

In some examples, the target position data 610 can further include inertial data and/or orientation data 640 recorded by the IMU 418. The IMU 418 includes sensors such as an accelerator, a compass, a gyroscope, and other sensors configured to record inertial data. The inertial and/or orientation data 640 facilitates the tracking of the position of the electronic device 400, especially while the electronic device 400 is in motion in the field of view of the user. The electronic device 400 can use the inertial and/or orientation data 640 as additional data points to refine the determination of the target position data 610 (e.g., including the target position relative to the user's face).

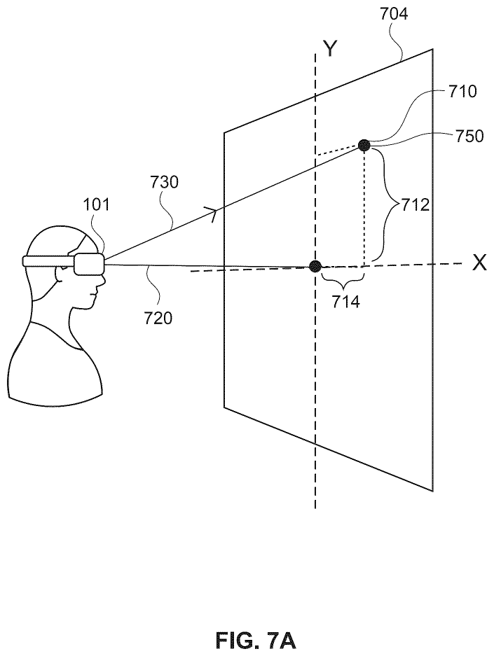

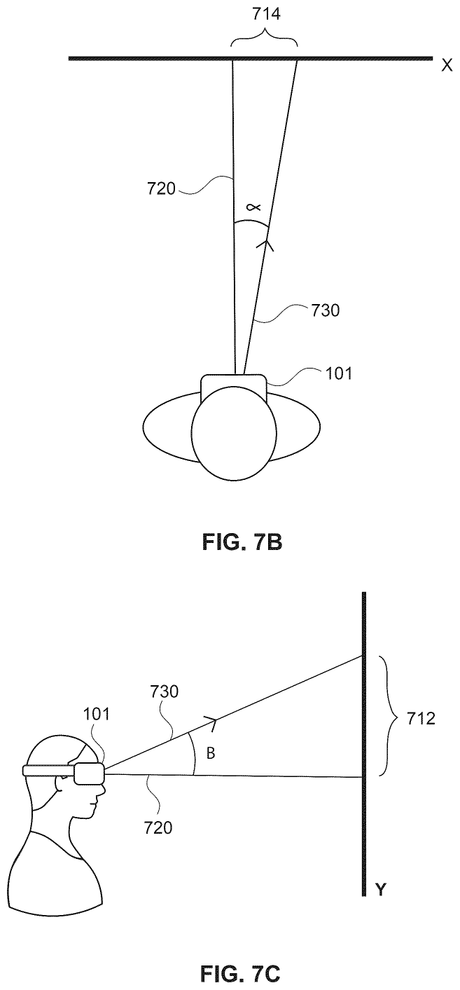

FIG. 7A-7C illustrate example representations of a position 710 of an exemplary object in the field of view 704 of the user according to an example of the disclosure. The position 710 of the object 750 is an example of position data relative to the head of the user as recorded by the electronic device 400 and/or the HMD 101. As such, the object 750 in these figures is merely an illustrative placeholder for a position 710 in space relative to the head of the user. As such, for example, the object 750 can be physical object such the electronic device 400, a virtual object such as the target 420, and/or a location in the field of view of the user such as the gaze point of user (e.g., a point in the field of view of the user that corresponds to the direction of gaze of the user). The position 710 of the object 750 is shown in a coordinate system. In some examples, the position 710 of the electronic device 400 can be determined relative to a reference based on the head of the user. For example, the centerline 720 of the user's face, which is an axis extending from a point centered between the eyes, can serve as the reference from which the position 710 of the object 750 relative to the user's face is expressed. As the centerline 720 of the user's face moves with the head of the user, the coordinate system based on the centerline 720 only represents the position of objects relative to the head of the user—and not relative to the physical world. In some examples, such as illustrated in FIGS. 7A-7C, the position 710 of the object 750 within the field of view of the user can be expressed in terms of azimuth 714 (e.g., a horizontal angle α from the centerline 720) and elevation 712 (e.g., a vertical angle β from the centerline 720). It is understood that other coordinate systems can be suitable for expressing the position 710 of an object relative to the head of the user. For example, the position 710 of the object 750 (e.g., the electronic device 400, the target 420, and/or gaze point of the user) relative to the head of the user can be expressed as cartesian coordinates having a reference point at the intersection of the centerline 720 with a field of view of the user. In some examples, the position of the object relative to the head of the user can be expressed using polar coordinates.

Referring back to FIG. 6, in some examples, the target position data 610 can include time stamps for recorded positions of the target 420. In some examples, the target position data 610 can include a time series of target positions relative to the user's face. In some examples, the target position data 610 can include a time code. In some examples, one or more of the component data streams of the target position data 610 (e.g., the electronic device position data 620, the target location data 630 on the display, and the inertial and/or orientation data 640) can include time stamps or timecodes. For example, the electronic device position data 620 as determined by the electronic device 400 based on facial recognition applied to the one or more images captured by the camera of the electronic device 400 can include time stamps and/or a timecode. In some examples, the target location data 630 based on the known position of the target on the display 412 can include time stamps and/or a timecode. In some examples, the inertial and orientation data 640 captured by the IMU 418 can include one or more timecodes (or in some examples, readings from the accelerator, the gyroscope, the compass, and other components of the IMU 418 can include timecodes).

The time stamps and/or timecodes of the target position data 610 can enable a processor (e.g., processor(s) 218A of HMD 101 such as described in reference to FIG. 2A) to synchronize target position relative to the user as captured during the motion of the target 420 with the direction of gaze of the user (also optionally time stamped or including a timecode, as described further below in relation to FIG. 9) as captured by the HMD 101 during the relative motion of the target 420. For example, the processor can match and/or cross-reference the time stamps (e.g., within the time codes) of data tracked by the electronic device 400 (e.g., the target position data 610 including the electronic device position data 620, the target location data 630 (e.g., target location on display 412), and the inertial data and/or orientation data 640), as well as time stamps of the data captured by the HMD 101, such as the direction of gaze of the user and other data (e.g., HMD inertial data and the HMD target position, to be further described below in reference to FIG. 9). In particular, the processor can match and/or cross-reference the time stamps (e.g., within the timecodes) of the target position data 610 with the time stamps (e.g., within the timecode) of the direction of gaze to determine the direction of gaze corresponding to each time stamp. Synchronizing the target position with the gaze direction as captured during the relative motion of the target 420 (e.g., synchronizing target movement with eye movement) can enable the processor to determine relationships between the target position and the gaze direction to evaluate the user's eye movement and detect the presence of abnormal eye movement.

In some examples, the target position data 610 can be stored on the electronic device 400 during the motion of the target relative to the head of the user. In some examples, the electronic device 400 can transmit the target position data to HMD 101 and/or another electronic device for processing during the movement of the electronic device 400 using communication circuitry 422.

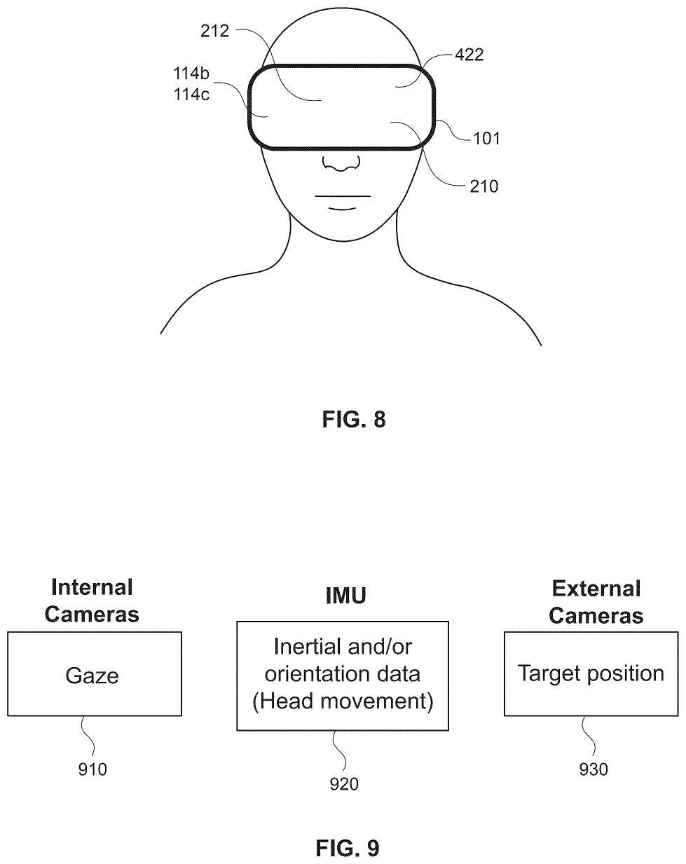

FIG. 8 illustrates an example of an HMD 101 according to an example of the disclosure. In some examples, HMD 101 corresponds to electronic device 201 described above with reference to FIG. 2A. The HMD 101 can include one or more eye tracking sensors 212 configured to track the user's eye movement during the vision test. In particular, the one or more eye tracking sensors 212 are configured to track the direction of the gaze of the user while the user's eyes are following (or eye is following) the target 420 as it moves in the field of view of the user. The HMD 101 can record the tracked direction of gaze of the user as the target 420 moves relative to the head of the user. In some examples, HMD 101 can track the direction of gaze using the eye tracking sensors 212 comprising at least one tracking camera and/or illumination source pointed toward the user's eyes, as previously described in reference to FIG. 2A. The eye tracking sensors 212 can record the gaze direction of one eye or two eyes. In some examples, the HMD 101 can capture the user's eye movement (e.g., the direction of the gaze of the user) while head of the user is stationary and the user's eyes follow the target 420 as it moves within the field of view of the user. In some examples, the HMD 101 can capture the movement of the user's eyes both when the eyes are looking within the field of view of the HMD 101 or outside of the field of view of the HMD 101. In some examples, the HMD 101 can capture the user's eye movement while the user's eyes are maintained on a stationary target 420 and the head of the user is moving relative to the stationary target. In some examples, the HMD 101 can capture the user's eye movement while both the head of the user and the target 420 are in motion.

The HMD 101 can further include further include motion and/or orientation sensor(s) 210A, such as an inertial measurement unit (IMU). The IMU can include or correspond to orientation sensor(s) 210A for detecting orientation and/or movement of HMD 101, such as previously described in reference to electronic device 201 in FIG. 2A. For example, the HMD 101 uses orientation sensor(s) 210A to track changes in the position and/or orientation of HMD 101, such as with respect to physical objects in the real-world environment. Orientation sensor(s) 210A optionally include one or more gyroscopes and/or one or more accelerometers.

The HMD 101 can further include one or more image sensors 206A. Unlike the one or more sensors configured to track eye movement, the one or more image sensors 206A can be outward-facing cameras configured to record the field of view of the user. In particular, the image sensors 206A are configured to track the position of the electronic device 400 and/or the target 420 as it moves relative to the user in the field of view of the user.

HMD 101 can further include communication circuitry 222A, such as previously discussed in reference to electronic device 201 and FIG. 2A. In some examples, HMD 101 can be in wired or wireless communication with electronic device 310 using communication circuitry 222A. The HMD 101 can employ the communication circuitry to receive target position data 610 and/or components thereof from the electronic device 400 (e.g., electronic device position data 620, target location data 630, and/or inertial data and/or orientation data 640 as previously described in reference to FIG. 6). The HMD 101 can receive the target position data 610 concurrently during the test (e.g., as live updates of the target position during the test). In some examples, the HMD 101 can receive the target position data 610 as recorded by electronic device after the test. The HMD can also transmit HMD data (e.g., such as discussed in reference FIG. 8) during or after the test.

FIG. 9 illustrates example representations of the data recorded by the HMD 101 during the test or the HMD data according to an example of the disclosure. In some examples, the one or more eye-tracking sensors or internal cameras can track the eye movement of the user as the user's eyes are following the target. Specifically, the internal cameras can track the direction of gaze of the user, which the HMD 101 can record as direction of gaze data 910. The direction of the gaze of the user can be recorded relative to a reference based on the head of the user, in some examples. Referring back to FIGS. 7A-C, the direction of the gaze of the user is shown as line 730 originating from the user's eyes. In some examples, the centerline 720 of the user's face can constitute the reference for the direction of the gaze of the user. As with the position 710 of an object in reference to the user's face as described in FIGS. 7A-C, the direction of the gaze of the user can be expressed in terms of azimuth 714 (e.g., a horizontal angle alpha from the centerline) and elevation 712 (e.g., a vertical angle beta from the centerline). It is understood that other coordinate systems can be suitable for expressing the direction of gaze. The HMD 101 can further determine the gaze point of the gaze of the user (e.g., the point in the user's field that corresponds to the gaze of the user) based on the direction of gaze. In FIGS. 7A-7C, the gaze point would correspond to position 710.

In some examples, the HMD 101 data can further include HMD inertial and/or orientation data 920 recorded by the IMU. When the HMD 101 is worn, the HMD inertial and/or orientation data 920 can correspond to or reflect the movement of head of the user (e.g., the HMD inertial data is head movement data). The tracked head movement (e.g., inertial and/or orientation data 920) facilitates the tracking of the direction of gaze, especially while the head of the user moves. The HMD 101 can use the head movement data (HMD inertial and/or orientation data 920) as additional data points to refine the tracked direction of gaze data 910. In some examples, system 300 can present instructions to the user based on the tracked head movement (e.g., as represented by HMD inertial and/or orientation data 920). For example, the HMD 101 (through a displayed message or visual indicator, or through an audible message) can instruct the user to keep their head still based on detecting from the HMD inertial and/or orientation data 920 that the head of the user has moved by a threshold amount (e.g., a threshold distance). In some examples, the HMD 101 can instruct the user to move their head in a particular manner (e.g., orientation, direction) during the test, for example while keeping the target 420 stationary. The instructions to the user can be output using one or more output devices such as the one or more speakers 216A of the HMD 101.

In some examples, the HMD data can further include the HMD target position data 930 as tracked by the external cameras of the HMD 101. The HMD target position data 930, which includes the position of the target 420 displayed on the electronic device as recorded by the HMD's external cameras, can be processed by the HMD 101 to refine the target position data 610 recorded by the electronic device 400 (e.g., a combination of target location data 630, electronic device position data 620, and/or inertial data and/or orientation data 640 as previously described in reference to FIG. 6). In some examples, such as in cases where the image sensors 206A (e.g., external cameras) of the HMD 101 track the electronic device 400 instead of the target 420 (e.g., when cameras cannot resolve the displayed target 420), the HMD target position data 930 can be the position of the electronic device.

In some examples, each of the direction of gaze data 910, the HMD inertial and/or orientation data 920, and the HMD target position data 930 can include time stamps and/or a timecode. For the direction of gaze data 910 for example, the timecode can enable a processor to match a direction of gaze to a particular time. The timecodes of the direction of gaze data 910, the HMD inertial and/or orientation data 920, and the HMD target position data 930 can be used synchronize these data streams to each other. The timecodes of the HMD data can further enable the synchronization of the direction of gaze data 910, the HMD inertial and/or orientation data 920, and the HMD target position data 930 with the target position data 610 recorded by the electronic device 400 (e.g., a combination of target location data 630, electronic device position data 620, and/or inertial data and/or orientation data 640 as previously described in reference to FIG. 6). Synchronizing the target position with the gaze direction as captured during the relative motion of the target (e.g., synchronizing target movement with eye movement) enable the processor to determine relationships between the target position and the gaze direction to evaluate the user's eye movement and detect the presence of abnormal eye movement.

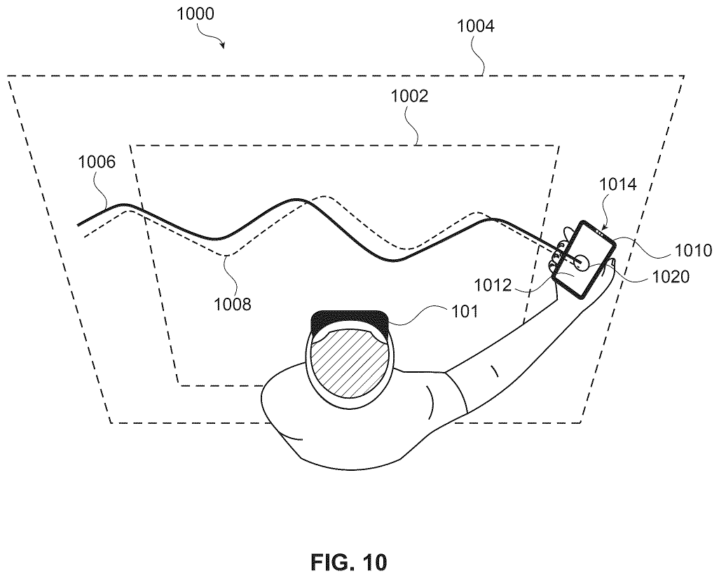

FIG. 10 illustrates an example of a user performing a vision test using system 1000 according to an example of the disclosure. System 1000 is similar to system 300, previously described. The user wears HMD 101 and holds an electronic device 1010 at a distance, with the display 1012 and camera 1014 of the electronic device 1010 oriented toward the face of the user. The electronic device 1010 displays a target 1020 that is visible to the user. The user moves the electronic device 1010 from one end of the field of view 1004 of the user to the other end (and through the field of view 1002 of HMD 101), causing the target 1020 to move along trajectory 1006 in the field of view 1004 of the user.

In some examples, system 1000 can present one or more instructions to guide the movement of the electronic device 1010 by the user. In some examples, system 1000 can provide instructions as audio messages via speakers 216A of HMD 101. For example, HMD 101 can instruct the user to place the electronic device 1010 at a particular position (e.g., to the left of the field of view of the user and level with eye(s) of the user. The HMD 101 can instruct the user on the movement of the electronic device (e.g., to move the electronic device along a particular trajectory and/or according to a particular pattern).

While the target 1020 is in motion, the user follows the target across their field of view 1004 with their eye(s) (with minimal movement of their head). In some examples, the HMD 101 can instruct the user (e.g., via the one or more speakers 216A) to keep their head still, and may further generate an alert if the HMD 101 detects (e.g., using IMUs as previously described) head movement that exceeds a threshold amount (e.g., a threshold distance). During the motion of the electronic device 1010 in the field of view 1004, the electronic device 1010 can record the target position data 610 (described in reference to FIG. 6) by recording the target location data 630 on the display 1012, tracking the position of the electronic device (e.g., as electronic device position data 620), and recording the inertial and/or orientation data 640 of the electronic device. Concurrently, the HMD 101 can track the direction of gaze of the user (e.g., as gaze data 910 as described in reference to FIG. 9) as the user follows the target 1020 with their eyes. The trajectory 1008 of the gaze of the user is shown. In some examples, the electronic device 1010 can be in communication with the HMD 101 (using communication circuitry 222A, such as described in reference to FIG. 2A) and can transmit the target position data 610 compiled by the electric device to the HMD 101 while the target is in motion.

In some examples, the HMD 101 can process the target position data 610 recorded by the electronic device and transmitted to the HMD 101, and the eye-tracking data recorded by the HMD 101. For example, processor(s) 218A for the HMD 101 can process the target position data 610 and the direction of gaze data 910. The processing can include synchronizing the target position data 610 and the direction of gaze data 910 using their respective time stamps and/or timecodes. For example, the processor can match and/or cross-reference the time stamps (e.g., within the time codes) of data tracked by the electronic device 400 (e.g., the target position data 610 including the electronic device position data 620, the target location data 630 (e.g., reflecting the location of target 1020 on the display 1012), and the inertial and/or orientation data 640), as well as time stamps of the data captured by the HMD 101, such as the direction of gaze of the user and other data (e.g., HMD inertial data and the HMD target position). In particular, the processor can match and/or cross-reference the time stamps (e.g., within the timecodes) of the target position data 610 with the time stamps (e.g., within the timecode) of the direction of gaze to determine the direction of gaze corresponding to each time stamp. Synchronizing the target position with the gaze direction as captured during the relative motion of the target (e.g., synchronizing target movement with eye movement) enables the processor to determine relationships between the target position and the gaze direction to evaluate the user's eye movement and detect the presence of abnormal eye movement.

In some examples, the processing can further include determining a relationship between the target position and the direction of gaze. For example, processor(s) 218A can conduct a comparison between the trajectory of the target in the field of view of the user and the trajectory of the direction of gaze (e.g., trajectory of the gaze point) in the field of view of the user to evaluate how well the direction of gaze matches the position of the target. In some examples, processor(s) 218A can determine that gaze trajectory matches the target trajectory (e.g., differences between the trajectories are less than a threshold angle), which can indicate that the eye movement is normal. In some examples, processor(s) 218A can determine that detected disparities between gaze trajectory and target trajectory are sufficiently large (e.g., exceed a threshold angle) to indicate abnormal eye movement. The processing of the target position data and eye tracking data can further include detecting characteristics and/or patterns in the gaze trajectory that indicate abnormal eye movement. While the processing of the target position data and eye tracking data is described as conducted by processor(s) 218A of HMD 101, it is understood that the target position data and the eye tracking data can be processed by other processors. For example, one or more processors of the electronic device 400 can process the target position data and the eye tracking data. In some examples, the target position data and the eye tracking data can be processed by one or more processors of a third-party device.

In some examples, HMD 101 can perform one or more actions in response to detecting the presence of abnormal eye movement and/or the absence of abnormal eye movement. In some examples, processor(s) 218A can perform the one or more actions, such as generating an indication of abnormal eye movement and/or normal eye movement. For example, processor(s) 218A can display information related to the processing (e.g., via display 120) which can include an indication of abnormal eye movement and/or normal eye movement. In some examples, processor(s) 218A can generate a notification related to the processing, which can include the indication of abnormal eye movement and/or normal eye movement as a notification. In some examples, a notification can be displayed indicating the possibility of abnormal eye movement. In some examples, the user can be prompted to take one or more actions including seeking out a medical professional for diagnosis. Additionally or alternatively, in some examples, the user can receive feedback including audio feedback and/or haptic feedback about eye movement or health. The processor(s) 218A can also transmit, when authorized by the user, via communication circuitry 222A, information related to the processing (e.g., including an indication of abnormal eye movement and/or normal eye movement) to the electronic device or to a third-party device. In some examples, the result (and a report including data) can be shared automatically (or manually) with the medical professional or with a health application.

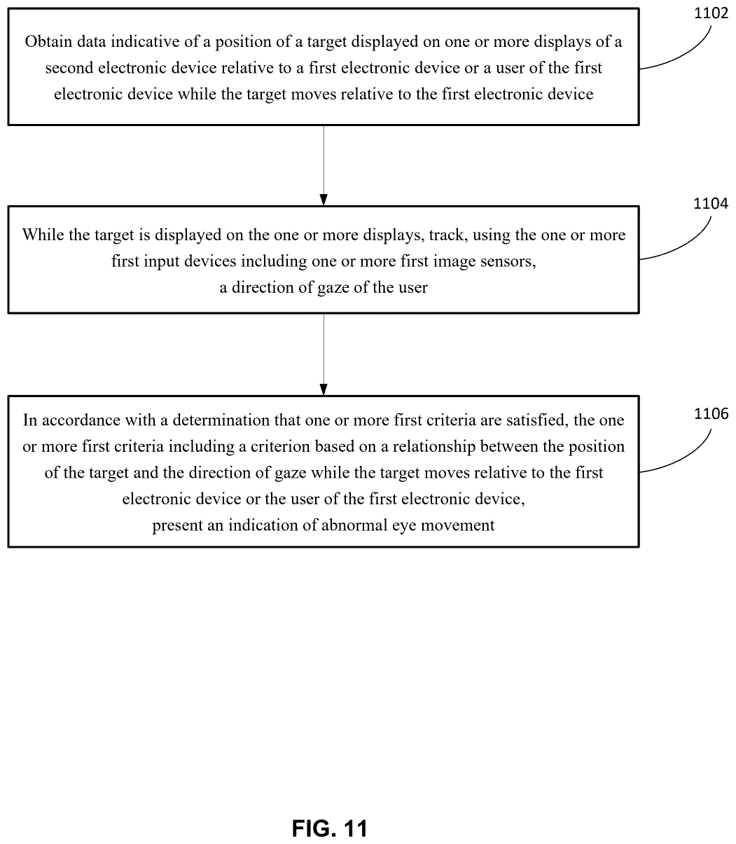

FIG. 11 illustrates an example flowchart of a method 1100 of performing a vision test according to an example of the disclosure. In some examples, method 1100 begins at a first electronic device in communication with one or more first input devices and in communication with a second electronic device including one or more displays. In some examples, the first electronic device is optionally a head mounted display similar or corresponding to device 201 of FIG. 2A, and the second electronic device is a mobile device similar or corresponding to device 400 of FIG. 4. In some examples, the first electronic device is a head mounted device without a display. As shown in FIG. 11, in some examples, the electronic device obtains (1102) data indicative of a position of a target displayed on the one or more displays of the second electronic device relative to the first electronic device or a user of the first electronic device while the target moves relative to the first electronic device in accordance with the examples described above. For example, the first electronic device (e.g., HMD 101) can obtain data indicative of a position of a target 320 displayed on display 312 of second electronic device 310, such as shown in FIGS. 3A and 3B, where the second electronic device 310 including the target 320 is shown moving relative to the first electronic device (e.g., HMD 101).

In some examples, while the target is displayed on the one or more displays, the first electronic device tracks (1104), using the one or more first input devices including one or more first image sensors, a direction of gaze of the user. For example, the first electronic device (e.g., HMD 101) can track a direction of gaze of the user (e.g., as shown by line 730 as in FIGS. 7A-7C) using one or more first image sensors such as camera 314 in FIGS. 3A-3B or eye tracking sensors 212 in FIGS. 2 and 8.

In some examples, the first electronic device or second electronic device processes the data indicative of the position of the target and the direction of gaze. For example, the first electronic device (e.g., HMD 101) can process the target position data 610 (e.g., the electronic device position data 620, the target location data 630 on the display, and the inertial and/or orientation data 640) such as shown in FIG. 6 and direction of gaze data 910 (as well as optionally, the HMD inertial and/or orientation data 920 and HMD target position data 930) such as shown in FIG. 9. In some examples, the second electronic devices can process the data indicative of the position of the target and the direction of gaze. In some examples, the data indicative of the position of the target and the direction of gaze can be processed by a third device.

In some examples, in accordance with a determination that one or more first criteria are satisfied, the one or more first criteria including a criterion based on a relationship between the position of the target and the direction of gaze while the target moves relative to the first electronic device or the user of the first electronic device, the first electronic device or second electronic device presents (1106) an indication of abnormal eye movement.

It is understood that process 1100 is an example and that more, fewer, or different operations can be performed in the same or in a different order. Additionally, the operations in process 1100 described above are, optionally, implemented by running one or more functional modules in an information processing apparatus such as general-purpose processors (e.g., as described with respect to FIGS. 2A-2B) or application specific chips, and/or by other components of FIGS. 2A-2B.

Therefore, according to the above, some examples of the disclosure are directed to a method including, at a first electronic device in communication with one or more first input devices and in communication with a second electronic device including one or more displays: obtaining data indicative of a position of a target displayed on the one or more displays of the second electronic device relative to the first electronic device or a user of the first electronic device while the target moves relative to the first electronic device; while the target is displayed on the one or more displays, tracking, using the one or more first input devices including one or more first image sensors, a direction of gaze of the user; and in accordance with a determination that one or more first criteria are satisfied, the one or more first criteria including a criterion based on a relationship between the position of the target and the direction of gaze while the target moves relative to the first electronic device or the user of the first electronic device, presenting an indication of abnormal eye movement. Additionally or alternatively to one or more of the examples disclosed above, in some examples, the data indicative of the position of the target can be based at least in part on one or more images of the first electronic device or the user of the first electronic device captured by one or more second image sensors of the second electronic device while the target moves relative to the first electronic device. Additionally or alternatively to one or more of the examples disclosed above, in some examples, the data indicative of the position of the target can be based at least in part on inertial data captured by one or more orientation sensors of the second electronic device while the target moves relative to the first electronic device. Additionally or alternatively to one or more of the examples disclosed above, in some examples, the one or more images of the first electronic device captured by the one or more second image sensors of the second electronic device can include one or more physical features of the first electronic device, the data indicative of the position of the target including a position of the second electronic device determined by the second electronic device based on the one or more physical features of the first electronic device. Additionally or alternatively to one or more of the examples disclosed above, in some examples, the one or more images of the user of the first electronic device can include identified features of a face of the user of the first electronic device in the one or more images of the first electronic device, the data indicative of the position of the target including a position of the second electronic device determined by the second electronic device based on the identified features of the face of the user of the first electronic device. Additionally or alternatively to one or more of the examples disclosed above, in some examples, the method can further include tracking an orientation of the first electronic device using one or more orientation sensors of the first electronic device, and processing the data indicative of the position of the target and the direction of gaze by processing data from the one or more orientation sensors of the first electronic device that is indicative of the orientation of the first electronic device. Additionally or alternatively to one or more of the examples disclosed above, in some examples, the data from the one or more orientation sensors of the first electronic device can include data obtained while the first electronic device is stationary and while the target displayed on the one or more displays of the electronic device moves relative to the user of the first electronic device. Additionally or alternatively to one or more of the examples disclosed above, in some examples, the data from the one or more orientation sensors of the first electronic device can include data obtained while the first electronic device moves and while the second electronic device is stationary. Additionally or alternatively to one or more of the examples disclosed above, in some examples, obtaining data indicative of the position of the target can include tracking the position of the second electronic device using one or more second input devices of the first electronic device while the target moves relative to the first electronic device. Additionally or alternatively to one or more of the examples disclosed above, in some examples, the method can further include synchronizing the data indicative of the position of the target and the direction of gaze. Additionally or alternatively to one or more of the examples disclosed above, in some examples, the method can further include detecting movement of the first electronic device using one or more sensors of the first electronic device; and in accordance with a determination that the movement is greater than a threshold amount, presenting, via one or more output devices of the first electronic device, instructions for keeping the first electronic device stationary. Additionally or alternatively to one or more of the examples disclosed above, in some examples, the method can further include presenting, via one or more output devices of the first electronic device, instructions for moving the second electronic device according to a pattern. Additionally or alternatively to one or more of the examples disclosed above, in some examples, the target can be a moving indicator displayed by the one or more displays of the second electronic device, wherein the moving indicator moves along an area of the one or more displays. Additionally or alternatively to one or more of the examples disclosed above, in some examples, the method can further include in accordance with a determination that a distance of the second electronic device from the first electronic device is a first distance, displaying the target on second electronic device at a first size, and in accordance with a determination that the distance of the second electronic device from the first electronic device is a second distance, different from the first distance, displaying the target on the second electronic device at a second size, different from the first size, wherein the first size and the second size are configured to maintain a constant angular size of the target from a perspective of the first electronic device. Additionally or alternatively to one or more of the examples disclosed above, in some examples, obtaining data indicative of the position of the target can includes obtaining data indicative of a first position of the target relative to the first electronic device or the user of the first electronic device while the target moves relative to the first electronic device; and obtaining data indicative of a second position of the target, different from the first position of the target, relative to the first electronic device or the user of the first electronic device while the target moves relative to the first electronic device. Additionally or alternatively to one or more of the examples disclosed above, in some examples, the first electronic device can further include one or more third image sensors having a first field of view, and a display visible to the user of the first electronic device and corresponding to the first field of view, and wherein the first position of the target is located within the first field of view and the second position of the target is located outside of the first field of view. Additionally or alternatively to one or more of the examples disclosed above, in some examples, the method can further include presenting, via one or more output devices of the first electronic device, instructions for moving the second electronic device from the first position of the target within the first field of view to the second position of the target outside of the first field of view. Additionally or alternatively to one or more of the examples disclosed above, in some examples, the method can further include transmitting to the second electronic device or a third electronic device the indication of abnormal eye movement. Additionally or alternatively to one or more of the examples disclosed above, in some examples, the method can further include, in accordance with a determination that the one or more first criteria are not satisfied, presenting an indication of normal eye movement.

Some examples of the disclosure are directed to an electronic device, comprising: one or more processors; memory; and one or more programs stored in the memory and configured to be executed by the one or more processors, the one or more programs including instructions for performing any of the above methods.

Some examples of the disclosure are directed to a non-transitory computer readable storage medium storing one or more programs, the one or more programs comprising instructions, which when executed by one or more processors of an electronic device, cause the electronic device to perform any of the above methods.

Some examples of the disclosure are directed to an electronic device, comprising one or more processors, memory, and means for performing any of the above methods.

Some examples of the disclosure are directed to an information processing apparatus for use in an electronic device, the information processing apparatus comprising means for performing any of the above methods.

The present disclosure contemplates that in some examples, the data utilized can include personal information data that uniquely identifies or can be used to contact or locate a specific person. Such personal information data can include demographic data, content consumption activity, location-based data, telephone numbers, email addresses, twitter ID's, home addresses, data or records relating to a user's health or level of fitness (e.g., vital signs measurements, medication information, exercise information), date of birth, or any other identifying or personal information. Specifically, as described herein, one aspect of the present disclosure is tracking a user's biometric data.

The present disclosure recognizes that the use of such personal information data, in the present technology, can be used to the benefit of users. For example, personal information data can be used to display suggested text that changes based on changes in a user's biometric data. For example, the suggested text is updated based on changes to the user's age, height, weight, and/or health history.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data can be governed by federal and/or state laws, such as the Health Insurance Portability and Accountability Act (HIPAA); whereas health data in other countries can be subject to other regulations and policies and should be handled accordingly. Hence different privacy practices should be maintained for different personal data types in each country.

Despite the foregoing, the present disclosure also contemplates examples in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to enable recording of personal information data in a specific application (e.g., first application and/or second application). In addition to providing “opt in” and “opt out” options, the present disclosure contemplates providing notifications relating to the access or use of personal information. For instance, a user can be notified upon initiating collection that their personal information data will be accessed and then reminded again just before personal information data is accessed by the one or more devices.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification can be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

The foregoing description, for purpose of explanation, has been described with reference to specific examples. However, the illustrative descriptions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

What is claimed is:

1. A method comprising:
at a first electronic device in communication with one or more first input devices and in communication with a second electronic device including one or more displays:
obtaining data indicative of a position of a target displayed on the one or more displays of the second electronic device relative to the first electronic device or a user of the first electronic device while the target moves relative to the first electronic device;
while the target is displayed on the one or more displays, tracking, using the one or more first input devices including one or more first image sensors, a direction of gaze of the user; and
in accordance with a determination that one or more first criteria are satisfied, the one or more first criteria including a criterion based on a relationship between the position of the target and the direction of gaze while the target moves relative to the first electronic device or the user of the first electronic device, presenting an indication of abnormal eye movement.

2. The method of claim 1, wherein the data indicative of the position of the target is based at least in part on one or more images of the first electronic device or the user of the first electronic device captured by one or more second image sensors of the second electronic device while the target moves relative to the first electronic device.

3. The method of claim 1, wherein the data indicative of the position of the target is based at least in part on inertial data captured by one or more orientation sensors of the second electronic device while the target moves relative to the first electronic device.

4. The method of claim 1, further comprising:
tracking an orientation of the first electronic device using one or more orientation sensors of the first electronic device; and
processing the data indicative of the position of the target and the direction of gaze by processing data from the one or more orientation sensors of the first electronic device that is indicative of the orientation of the first electronic device.

5. The method of claim 1, wherein obtaining data indicative of the position of the target includes tracking the position of the second electronic device using one or more second input devices of the first electronic device while the target moves relative to the first electronic device.

6. The method of claim 1, further comprising:
detecting movement of the first electronic device using one or more sensors of the first electronic device; and
in accordance with a determination that the movement is greater than a threshold amount, presenting, via one or more output devices of the first electronic device, instructions for keeping the first electronic device stationary.

7. The method of claim 1, wherein the target is a moving indicator displayed by the one or more displays of the second electronic device, wherein the moving indicator moves along an area of the one or more displays.

8. The method of claim 1, wherein the method further comprises:
in accordance with a determination that a distance of the second electronic device from the first electronic device is a first distance, displaying the target on the second electronic device at a first size; and
in accordance with a determination that the distance of the second electronic device from the first electronic device is a second distance, different from the first distance, displaying the target on the second electronic device at a second size, different from the first size, wherein the first size and the second size are configured to maintain a constant angular size of the target from a perspective of the first electronic device.

9. A first electronic device comprising:
a memory;
one or more first input devices;
one or more processors; and
one or more programs stored in the memory and configured to be executed by the one or more processors, the one or more programs including instructions for performing a method comprising:
obtaining data indicative of a position of a target displayed on the one or more displays of a second electronic device relative to the first electronic device or a user of the first electronic device while the target moves relative to the first electronic device;
while the target is displayed on the one or more displays, tracking, using the one or more first input devices including one or more first image sensors, a direction of gaze of the user; and
in accordance with a determination that one or more first criteria are satisfied, the one or more first criteria including a criterion based on a relationship between the position of the target and the direction of gaze while the target moves relative to the first electronic device or the user of the first electronic device, presenting an indication of abnormal eye movement.

10. The first electronic device of claim 9, wherein the data indicative of the position of the target is based at least in part on one or more images of the first electronic device or the user of the first electronic device captured by one or more second image sensors of the second electronic device while the target moves relative to the first electronic device.

11. The first electronic device of claim 9, wherein the data indicative of the position of the target is based at least in part on inertial data captured by one or more orientation sensors of the second electronic device while the target moves relative to the first electronic device.

12. The first electronic device of claim 9, wherein the method further comprises:
tracking an orientation of the first electronic device using one or more orientation sensors of the first electronic device; and
processing the data indicative of the position of the target and the direction of gaze by processing data from the one or more orientation sensors of the first electronic device that is indicative of the orientation of the first electronic device.

13. The first electronic device of claim 9, wherein obtaining data indicative of the position of the target includes tracking the position of the second electronic device using one or more second input devices of the first electronic device while the target moves relative to the first electronic device.

14. The first electronic device of claim 9, wherein the method further comprises:
detecting movement of the first electronic device using one or more sensors of the first electronic device; and
in accordance with a determination that the movement is greater than a threshold amount, presenting, via one or more output devices of the first electronic device, instructions for keeping the first electronic device stationary.

15. The first electronic device of claim 9, wherein the target is a moving indicator displayed by the one or more displays of the second electronic device, wherein the moving indicator moves along an area of the one or more displays.

16. The first electronic device of claim 9, wherein the method further comprises:
in accordance with a determination that a distance of the second electronic device from the first electronic device is a first distance, displaying the target on second electronic device at a first size; and
in accordance with a determination that the distance of the second electronic device from the first electronic device is a second distance, different from the first distance, displaying the target on the second electronic device at a second size, different from the first size, wherein the first size and the second size are configured to maintain a constant angular size of the target from a perspective of the first electronic device.

17. A non-transitory computer readable storage medium storing one or more programs, the one or more programs comprising instructions, which when executed by one or more processors of a first electronic device, cause the first electronic device to perform a method comprising:
obtaining data indicative of a position of a target displayed on the one or more displays of a second electronic device relative to the first electronic device or a user of the first electronic device while the target moves relative to the first electronic device;
while the target is displayed on the one or more displays, tracking, using one or more first input devices including one or more first image sensors, a direction of gaze of the user; and
in accordance with a determination that one or more first criteria are satisfied, the one or more first criteria including a criterion based on a relationship between the position of the target and the direction of gaze while the target moves relative to the first electronic device or the user of the first electronic device, presenting an indication of abnormal eye movement.

18. The non-transitory computer readable storage medium of claim 17, wherein the data indicative of the position of the target is based at least in part on one or more images of the first electronic device or the user of the first electronic device captured by one or more second image sensors of the second electronic device while the target moves relative to the first electronic device.

19. The non-transitory computer readable storage medium of claim 17, wherein the data indicative of the position of the target is based at least in part on inertial data captured by one or more orientation sensors of the second electronic device while the target moves relative to the first electronic device.

20. The non-transitory computer readable storage medium of claim 17, wherein the method further comprises:
tracking an orientation of the first electronic device using one or more orientation sensors of the first electronic device; and
processing the data indicative of the position of the target and the direction of gaze by processing data from the one or more orientation sensors of the first electronic device that is indicative of the orientation of the first electronic device.

21. The non-transitory computer readable storage medium of claim 17, wherein obtaining data indicative of the position of the target includes tracking the position of the second electronic device using one or more second input devices of the first electronic device while the target moves relative to the first electronic device.

22. The non-transitory computer readable storage medium of claim 17, wherein the method further comprises:
detecting movement of the first electronic device using one or more sensors of the first electronic device; and
in accordance with a determination that the movement is greater than a threshold amount, presenting, via one or more output devices of the first electronic device, instructions for keeping the first electronic device stationary.

23. The non-transitory computer readable storage medium of claim 17, wherein the target is a moving indicator displayed by the one or more displays of the second electronic device, wherein the moving indicator moves along an area of the one or more displays.

24. The non-transitory computer readable storage medium of claim 17, wherein the method further comprises:
in accordance with a determination that a distance of the second electronic device from the first electronic device is a first distance, displaying the target on second electronic device at a first size; and
in accordance with a determination that the distance of the second electronic device from the first electronic device is a second distance, different from the first distance, displaying the target on the second electronic device at a second size, different from the first size, wherein the first size and the second size are configured to maintain a constant angular size of the target from a perspective of the first electronic device.
